(19) United States
(12) Patent Application Publication
(10) Pub. No.: US 2011/0202018 A1
(43) Pub. Date: Aug. 18, 2011

(54) SLEEVE FOR TRANSDERMAL PATCHES

(76) Inventors: Tilak Kithsiri Dias, Stockport (GB); William Hunter, Cheshire (GB)

(21) Appl. No.: 13/056,328

(22) PCT Filed: Aug. 3, 2009
(86) PCT No.: PCT/GB2009/001903

§ 371 Date: May 3, 2011

(30) Foreign Application Priority Data
Aug. 1, 2008 (GB) 0814152.5

(51) Int. Cl. A61M 35/00; D04B 35/00

(52) U.S. Cl. 604/290; 604/308; 66/147

(57) ABSTRACT

A contact element is held against the skin of a human limb by a sleeve comprising a tube body (2) knitted with elastomeric yarn. At the ends of the tube body margins (4) are formed which prevent the tube from rolling on itself. In preferred embodiments the margins 4 are also knitted, with a different knitting pattern from that used in the tube body, which is normally a plain knitted structure. Sleeves of the invention can be manufactured in a tubular knitting process on a computerised flat bed knitting machine.

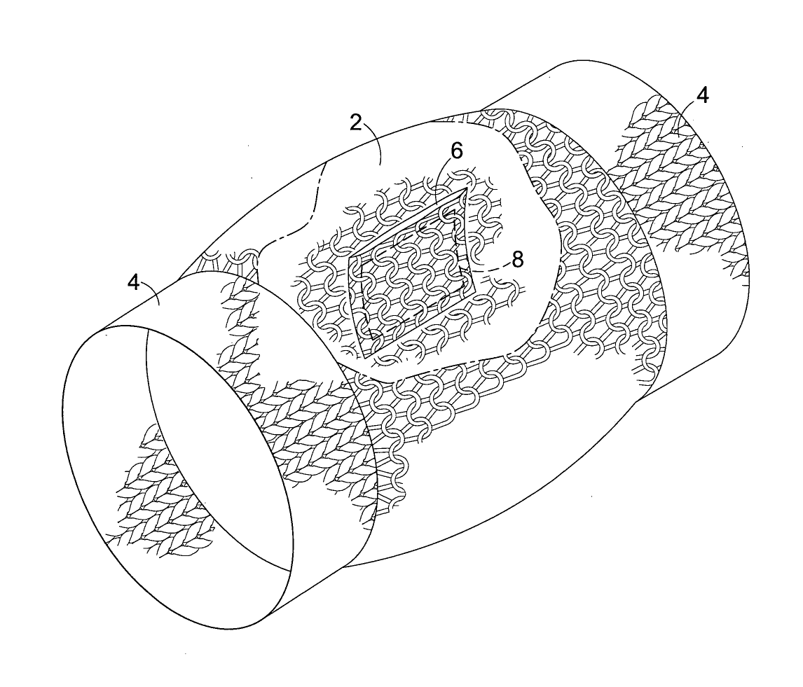

This invention relates to dermal patches, and particular to mechanisms and devices for holding them in place on the human body. Such patches have many uses, including ongoing wound treatment, drug delivery systems, and damage protection. This invention has particular, but not exclusive use in transdermal drug delivery systems. Devices for use in such systems are disclosed in International Patent Application No: PCT/GB2009/00132, incorporated by reference.

Traditionally, dermal patches have been held in place on the human skin by a temporary adhesive bond. Such bonds can be uncomfortable for the user, and painful to break when the patch has to be removed. They can also provoke reactions in sensitive skin. Dermal patches are prone to accidental removal due to local conditions created by for example excessive heat, moisture and abrasion. In the case of drug delivery in particular, it is essential that the patch stays in place for the prescribed period. The present invention seeks to provide an alternative technique for holding such patches in place. It may also be used to keep adhesive dermal patches in place even when the adhesive fails.

In the medical field elastic, sleeves have been proposed for covering wound or intravenal needle sites, or as external covers for support devices. U.S. Pat. No. 5,836,904 discloses an integral sleeve and cover for a medical support device comprising a sleeve body formed substantially of cotton and elastomeric fibres. The body has a lining and a cover portion. The lining portion is wrapped around the wearer's limb, the support device disposed over the lining, and the cover portion then wrapped around the limb to cover the support device. International Patent Specification No: WO 2007/064,885 discloses a wound cover in the form of an elastic panel for wrapping around a body part to cover a wound site and secure padding thereover. Opposite sides of the panel form a reclosable fastener system enabling the cover to be easily applied.

According to the present invention, a contact element is held against the skin of a human limb by a sleeve comprising a tube body knitted with elastomeric yarn. The yarns provide resiliency to hold the tube against the skin with pressure determined by the structure of the tube and of course the relation between the dimensions of the tube and the human limb. The pressure can thus be predicted, and therefore selected, by matching the sleeve to the wearer, to ensure sufficient pressure is maintained to hold a contact element in place in the circumstances in which it is to function. At the ends of the tube margins are formed which prevent the tube from rolling on itself. Such margins can be created by simple reinforcement; by different structures coupled to the tube body; or the adoption of a different knitting pattern from that used in a tube body, which is normally a plain knitted structure. In a preferred embodiment, the tube ends comprise a rib knitted structure with elastomeric yarn, which can be the same elastomeric yarn that forms the tube itself. It is preferred that the body of the tube between the margins is knitted only from elastomeric yarns.

As noted above, tubular plain jersey knitting is normally used for the tube body. Knitted margins can be rib or purl structure, with 1×1 or 2×2 rib being preferred. Rib and purl structures are considered as balanced and are not inclined to edge curl. Plain knitting is produced by the knitting needles moving in a single plane; thus all the knitted loops are interloped in the same direction. Rib knitting requires two sets of needles operating in between each other in two different needle planes so that the knitted loops in neighbouring wales are interloped in opposite directions. In purl knitting the neighbouring courses are interloped in opposite directions. When a rib structure is used in the margins of a sleeve according to the invention, one or more, preferably two courses of purl knitting may be included between at least one margin and the tube body. When the sleeve is used on a wearer's upper arm, around the biceps, such an insertion adjacent the top or upper margin assists in preventing downward slippage, while also explaining the orientation in which the sleeve is to be worn.

The yarns used in the body of the tube may be multifilament or monofilament elastomeric yarns. Monofilament yarns have the advantage of having very little capacity for liquid retention in a knitted structure. This is a valuable asset for products to be used close to the human skin. Preferred materials for the elastomeric yarns; monofilament or multifilament, are polyurethane, rubber and silicone. These yarns, and particularly such monofilament yarns, assist in carrying liquid, such as perspiration, away from the surface upon which the sleeve is mounted. Suitable elastomeric yarns are available from Asahi Kasei Group under the Trade Mark ROICA, and from Investa Corporation under the Trade Mark LYCRA. In a particularly preferred embodiment of the invention, the tube body is knitted with monofilament yarns, and the margins with multifilament elastomeric yarns.

In sleeves according to the invention the margins can be knitted with double covered elastomeric yarns. A double covered yarn is one in which two strands of a non-elastic covering yarn are wound in opposite directions around a core elastomeric yarn. The core may be monofilament or multifilament. Double covered yarns are particularly useful in the present invention as they provide additional resistance to rolling or furling of the sleeve margins.

The contact elements to be held against the human skin by a sleeve according to the invention as set out above, have a variety of uses. One is a transducer, and for such an application the sleeve can include at least one elastomeric yarn carrying a conductive filament for conduction to such a transducer element. One or more yarns may also carry other materials useful in the protection or treatment of the skin to which the sleeve is applied. For example, a yarn could carry an antimicrobial coil wound around it. Sleeves of the invention can be designed to achieve an optimum balance between grip, element retention and comfort in particular circumstances.

The contact element to be held against the skin according to the invention can be included in the sleeve, and in some embodiments integrally knitted into the tube structure. Alternatively, the sleeve could be formed with a pocket in the tube for carrying the contact element. The contact element could be a transdermal treatment patch, and provision can be made for enabling the contact element or elements to be charged or recharged with a treatment substance while it is in situ in the tube and against a skin surface. This would be appropriate particularly in situations where the sleeve is being used in a treatment in which a substance is or is to be progressively released from the contact element(s) to the skin.

While the sleeve of the invention has been described above for holding a contact element against the skin of a human limb, it can also be used in association with other body parts if suitably adapted. Thus, the sleeve can be made as a sheet or strip with attachable ends. The ends can be adapted to overlap, than the overall circumference of the created sleeve can be adjusted to suit a particular application. This enables the same sleeve to be used on patients' limbs of different dimensions or where it is adapted to be wrapped around the human torso, it can be adapted to suit individual patients, or different parts of individual patients. The important feature of the sleeves according to the invention is of course that the pressure they apply to hold a contact element in place against the skin is predictable and can be determined in a particular situation.

Sleeves according to the invention can be made in different sizes to suit the limbs of different wearers. Generally, in its relaxed state, the ratio of the sleeve circumference at its midpoint to the circumference of each margin is in the range 1.0 to 1.5, preferably 1.2 to 1.4. The ratio of the overall axial sleeve length to the axial length of each margin is, with the sleeve relaxed, in the range 5.0 to 6.5 The preferred axial length of each margin is 25 mm. The sleeves are normally designed for use on a wearer's upper arm around the biceps to hold a transdermal patch in place. Typical dimensions (mm) of the relaxed sleeve, for three sizes are as follows.

| | |
|---|---|
| Circumference | Axial Length |

| Tube Body | Margin | Margin | Length | Length |
|---|---|---|---|---|
| Midpoint | Top | Bottom | Overall | Margin |
| 175 | 140 | 140 | 140 | 25 |
| 210 | 160 | 160 | 142 | 25 |
| 240 | 180 | 180 | 145 | 25 |

The invention will now be described by way of example and with reference to the accompanying drawing which is a broken perspective view of a sleeve according to the invention.

As shown in the drawing, the sleeve comprises a tube section 2 with a marginal portion 4 at either end. The tube portion 2 is knitted in a tubular plain knitting process on a flat-bed knitting machine which creates a seamless tube. In the embodiment shown the elastomeric yarns of the tube extend into the margins 4 where the knitting style is adapted to rib tubular structure to create the margins in a form which is curl-resistant, and prevent the tube from rolling up on itself from either end during its use. On a modern computerised flat-bed knitting machine the structure can be knitted either in half-gauge or full-gauge.

The sleeve is knitted on a computerised flat-bed knitting machine. The main body of the sleeve is knitted with an elastomeric monofilament yarn; two courses are knitted only on the front needle bed, and then the next two courses are knitted on the back needle bed. In order to create a tubular knitted fabric the selvedge wales of the two courses which were knitted on the two needle beds are connected with tuck loops. The above technique provides a balanced structure free from self twisting after knitting.

The top and bottom margins are produced with the rib knitted structure. Two sets of needles that are moving in two different needle planes are required to knit a rib or a purl knitted structure. In order to meet this requirement, the two margins are knitted on half gauge while the main body section is knitted on full gauge; i.e., every second needle is used for knitting. This leaves a set of needles free on each needle bed for transferring knitted loops between the two needle beds in order to knit a tubular rib and/or tubular purl knitted structure.

The monofilament elastomeric yarn is stretched to a very high degree, of the order of 600%, during the knitting process. After knitting the monofilament yarn has to be relieved of the stress energy introduced during the stitch formation process. This can be achieved with a steam table as is common practice in the knitwear industry. By placing a thin plastic former inside the sleeve prior to the steaming process a defined dimensional stability of the sleeve can be achieved.

Also shown in the drawing is a pocket 6 housing a contact element 8 which might be a transdermal patch providing controlled release of a drug or other substance onto the skin. The pocket is normally formed on the inside surface of the tube as shown, but can be on the external surface. Whichever style is adopted, there is layer of a tube fabric between the contact element and the skin. However, in some applications it is sufficient merely to locate the contact element on the skin, and apply the sleeve thereover. The resilience of the sleeve will hold the contact element in place, without a specific location of the contact element relevant to the tube being defined.

With the contact element in the pocket, it can be readily replaced. Alternatively, provision can be made for recharging it with treatment substance in situ. In such an application a contact element could be adapted to receive a treatment substance either through an appropriate union formed thereon or by injection from outside.

### EXAMPLE

A sleeve of the kind illustrated, but without the pocket was knitted on a flat bed knitting machine using ROICA HS 570dtex monofilament elastane fibre, available from Asahi Kasei Fibers Corporation in Japan for the tube body; and Wykes D963A, double covered composite yarn—ROICA HS 570dtex core with polyamide cover, available from Wykes International Limited in the United Kingdom for the margins. The knitting machine used was from Shima Seiki Mfg. Ltd in Japan; Model SES1225 with ten (10) needles per inch, and Meminger EFS700 positive yarn feed units for the delivery of elastane fibres.

Knitting commenced at the elbow or bottom end of the sleeve and finished at the shoulder or top end. A set-up is performed using needles on both needle beds, in a fashion that will allow the creation of a tubular knitted sleeve. A number of rows of 2×2 rib construction are knitted in tubular fashion, which prevents the armband from rolling back when worn. The main body rows are knitted in plain tubular fashion by knitting with all needles on alternating needle beds as described earlier. On completion of this section, all needles being knitted are transferred back to the 2×2 rib set out and the top ribbed (shoulder end) section is knitted. When the required number of courses has been knitted, a binding off (casting-off) process is performed by the knitting machine to securely lock the last row of knitted stitches and allow removal of the piece from the knitting machine.

Sleeves according to the above example have been found to be effective in holding self-adhesive (polyacrylate) patches on arms over an extended period relative to retention of the patches without the sleeve. Sleeves were used by volunteers over a period of three days, and the securement of each patch was checked daily before and after three hours of hard exercise in protective clothing at temperatures of around 38° C. All the patches held in place by sleeves remained in place throughout, while the securement of all the patches without the sleeve had deteriorated significantly. Furthermore, the sleeves remained in place, without retaining moisture, and without the margins furling. The sleeves remained comfortable throughout.

1. A sleeve for holding a contact element against the skin of a human limb, comprising a tube body knitted with elastomeric yarns between the tube ends, which tube ends form margins preventing rolling of the tube on itself.

2. A sleeve according to claim 1 wherein the elastomeric yarns are polyurethane yarns.

3. A sleeve according to claim 1 wherein the elastomeric yarns are silicone yarns.

4. A sleeve according to claim 1 wherein the elastomeric yarns are rubber yarns.

5. A sleeve according to claim 1 wherein the elastomeric yarns comprise monofilament yarns.

6. A sleeve according to claim 1 wherein the elastomeric yarns comprise multifilament yarns.

7. A sleeve according to claim 1 wherein the tube body is knitted with monofilament yarns and the margins comprise knitted multifilament yarns.

8. A sleeve according to any of claim 1 wherein the tube body is knitted with monofilament yarns and the margins comprise knitted double covered yarns.

9. A sleeve according to claim 1 for holding an electronic transducer element, and including at least one elastomeric yarn carrying a conductive filament for connection to a said element.

10. A sleeve according to claim 1 including at least one elastomeric yarn carrying an anti-microbial coil wound therearound.

11. A sleeve according to claim 1 wherein the tube body is plain knitted.

12. A sleeve according to claim 1 wherein the margins comprise rib structure knitted with elastomeric yarns.

13. A sleeve according to claim 12 wherein the rib structure is knitted with double covered elastomeric yarns.

14. A sleeve according to claim 12 including two courses of purl knitted structure between at least one margin and the tube body.

15. A sleeve according to claim 1 wherein the margins comprise purl structure knitted with elastomeric yarns.

16. A sleeve according to claim 15 wherein the purl structure is knitted with double covered elastomeric yarns.

17. A sleeve according to claim 1 wherein a pocket is formed in the tube for carrying a said contact element.

18. A sleeve according to claim 1 including a said contact element.

19. A sleeve according to claim 18 wherein the contact element is integrally knitted into the tube structure.

20. A sleeve according to claim 18 wherein the contact element is a transdermal treatment patch.

21. A sleeve according to claim 18 wherein the contact element is chargeable or re-chargeable with a treatment substance.

22. A sleeve according to claim 1 wherein in its relaxed state the ratio of the sleeve circumference at its midpoint to the circumference of each margin is in the range 1.0 to 1.5.

23. A sleeve according to claim 22 wherein said range is 1.2 to 1.4.

24. A sleeve according to claim 1 wherein in its relaxed state the ratio of the overall axial sleeve length to the axial length of each margin is in the range 5.0 to 6.5.

25. A sleeve according claim 1 wherein the axial length of each margin is 25 mm.

26. A sleeve according to claim 1 for use in transdermal therapy.

27. A method of manufacturing a sleeve according to claim 1 in a tubular knitting process commencing at one end of the sleeve.

28. A method according to claim 27 wherein the margins are knitted with a rib or purl structure and the tube body is in plain jersey knit.

29. A method according to claim 27 wherein the elastomeric yarn is extended in the knitting process, the method including the step of relieving the generated stress with a steam table.

30. A method according to claim 29 wherein a plastics former is placed inside the sleeve prior to the steaming process.

31. A method of holding a contact element against the skin of a human limb, comprising fitting the limb with a sleeve according to any of claim 1 with a contact element installed under the sleeve and against the skin.

32. A method of holding a contact element against the skin of a human limb, comprising fitting the limb with a sleeve according to claim 18.

33. The use of a sleeve according to claim 26 in a method of transdermal treatment in which the contact element is installed under the sleeve and against the skin, and a treatment substance is released from the contact element to the skin.

34. The use of a sleeve as set out in claim 33, including the step of charging or re-charging the contact element with a said treatment substance while the sleeve is fitted on a said limb with the contact element thereunder.
